(19) United States
(12) Patent Application Publication
(10) Pub. No.: US 2025/0213402 A1
(43) Pub. Date: Jul. 3, 2025

(54) SANITARY ARTICLE WITH ASSYMMETRICAL FASTENING WINGS AND METHODS OF ATTACHING A COMMON RELEASE PAPER TO THE FASTENING WINGS OF THE SANITARY ARTICLE

(71) Applicant: Essity Hygiene and Health Aktiebolag, Goteborg (SE)

(72) Inventors: Annika Dahl, GÖTEBORG (SE); Louise Eliasson, GÖTEBORG (SE)

(21) Appl. No.: 18/843,959

(22) PCT Filed: Apr. 1, 2022
(86) PCT No.: PCT/EP2022/058785

§ 371 Date: Sep. 4, 2024

(51) Int. Cl. A61F 13/56; A61F 13/15

(52) U.S. Cl. CPC A61F 13/5616; A61F 13/15756

(57) ABSTRACT

Disclosed is a sanitary article comprising a topsheet and a backsheet, the sanitary article extending in a longitudinal direction and in a transverse direction, the sanitary article having a first and second longitudinal side edges, a front edge and a rear edge, the sanitary article having a first fastening wing extending from the first longitudinal side edge and a second fastening wing extending from the second longitudinal side edge, the first fastening wing including a first coated adhesive region, being constituted by the total adhesive area provided on a garment facing side thereof and the second fastening wing including a second coated adhesive region, being constituted by the total adhesive area provided on a garment facing side thereof.

### CROSS REFERENCE TO RELATED APPLICATIONS

This application is a National Stage Application of PCT/EP2022/058785, filed Apr. 1, 2022, the contents of which are incorporated in their entirety herein.

### TECHNICAL FIELD

The present disclosure pertains to a sanitary article such as a sanitary napkin or a pantiliner including fastening wings and to methods of attaching a release paper to the fastening wings. In particular, this disclosure pertains to a sanitary article having asymmetrical fastening wings, the fastening wings being folded on top of the sanitary article and provided with a common release paper.

### BACKGROUND

A sanitary article of the kind to which this disclosure relates includes an absorbent core arranged between a topsheet and a backsheet, it further includes so-called fastening wings provided with adhesive regions for fastening of the wings in a user undergarment.

When using a sanitary article, the article should be designed to be comfortable to wear for the user and is should provide a high level of protection for the user undergarment and clothing. When wearing the sanitary article, the user often moves around which may cause the sanitary article to move as well. This may result in incorrect positioning of the sanitary article and undesired wrinkling and folding of the sanitary article, which may negatively affect comfort, absorption and protection of the user garments. To secure that the sanitary article remains in place, the size and extension of the fastening wings may be increased.

When manufacturing the sanitary articles and providing the fastening wings with adhesive regions, the fastening wings are conventionally folded onto the topsheet of the sanitary article. The adhesive regions provided on the fastening wings are protected by a release paper covering the adhesive regions and keeping the fastening wings in place during manufacturing, storage and handling of the sanitary article. To simplify the manufacturing, the adhesive regions may be covered by a common release paper extending over the adhesive regions of both the fastening wings.

Manufacturing of absorbent articles, such as sanitary articles are conventionally produced in high-speed production lines including the assembly of a plurality components, such as web materials and absorbent layers. For sanitary articles the production additionally includes the application of adhesive and the overlying release paper. All these components are applied, remain in place during manufacturing, and are fastened in a correct position of the article. For example, if the release paper is not correctly positioned or attached to the adhesive regions, edge portions may be caught in the air flow in the high-speed causing detached or folded release paper regions or folded fastening wings. This may result in an increased amount of defective products and undesired interruptions in the high-speed production lines, causing an inefficient production and an increased cost to manufacture the sanitary articles.

It is therefore desirable to offer an improved sanitary article and improvements in the method for producing the sanitary article and to reduce the number of defective articles resulting from the production.

### SUMMARY

One or more of the above objects may be achieved with a sanitary article in accordance with claim 1 or in accordance with any one of the methods for producing a sanitary article according to claim 15 or 16. Further embodiments are set out in the dependent claims, in the following description and in the drawings.

The present disclosure relates to a sanitary article, such as a sanitary napkin or a pantiliner, including a topsheet, and a backsheet. The sanitary article extends in a longitudinal direction and in a transverse direction and has a first and a second longitudinal side edge, a front edge, and a rear edge. The sanitary article has a first fastening wing extending from the first longitudinal side edge and a second fastening wing extending from the second longitudinal side edge, the first fastening wing including a first coated adhesive region, being constituted by the total adhesive area provided on a garment facing side thereof and the second fastening wing including a second coated adhesive region, being constituted by the total adhesive area provided on a garment facing side thereof. The first coated adhesive region has a first total surface area and the second coated adhesive region has a second total surface area. The first and the second fastening wings are arranged in a folded configuration onto the topsheet and the first and the second adhesive regions are coated adhesive regions being covered with a common release paper. The first and second fastening wings are asymmetrical with respect to each other, as seen along any longitudinal axis extending in the longitudinal direction, and the first and second coated adhesive regions are asymmetrical with respect to each other, as seen along any longitudinal axis extending in the longitudinal direction. A majority of the first surface area of the first coated adhesive region is arranged offset with respect to a majority of the second surface area of the second coated adhesive region, as seen in the longitudinal direction, and the common release paper has a release force value within the range of from 5 to 30 N/m as measured according to the Release Force Method as disclosed herein below.

The term “sanitary article” refers to a product that is placed against the skin of the wearer in the wearer's crotch to absorb and contain body exudates, like discharges, urine, faeces and menstrual fluid. The disclosure mainly refers to disposable sanitary articles, which means articles that are not intended to be laundered or otherwise restored or reused as a sanitary article. Examples of disposable sanitary articles include feminine hygiene products such as sanitary napkins, panty liners, incontinence pads, and the like.

The sanitary article may include an absorbent core arranged between the topsheet and the backsheet.

The fastening wings may be folded on top of the topsheet in a non-overlapping manner.

The asymmetry of the fastening wings of the sanitary napkin with respect to each other, as seen along any longitudinal axis, enables larger fastening wings having a greater extension, as seen in the transverse direction. During manufacturing, the fastening wings are folded on top of the topsheet, preferably in a non-overlapping manner, and preferably as close to the outer edges of the absorbent core as possible to improve packaging efficiency and stability. As the fastening wings of the sanitary article are asymmetric, as seen along any longitudinal axis, the width of the fastening wings may be larger than the width of the sanitary article without overlapping each other.

During manufacturing of the sanitary articles and after folding of the fastening wings on top of the topsheet the fastening wings are provided with adhesive on a garment facing side thereof and then the adhesive zones are subsequently covered with a common release paper. Alternatively, the common release paper may be provided with adhesive zones and the release paper may subsequently be added on top of the folded fastening wings to provide the fastening wings with respective adhesive zones. As the fastening wings are asymmetric, as seen along any longitudinal axis, and since the first and second coated adhesive regions are asymmetrical with respect to each other, as seen along any longitudinal axis extending in the longitudinal direction and a majority of the first surface area of the first coated adhesive region is arranged offset with respect to a majority of the second surface area of the second coated adhesive region, the common release paper may have greater portions at the front edge or rear edge (depending on the transport direction of the sanitary articles during manufacturing) being unattached to the fastening wings.

In the manufacturing process and when running the process on high-speed, unattached portions may be caught in the air flow in the machine, resulting in detachment or folding of the release paper, or folding of the fastening wing under the release paper. This may in turn result in increased number of defective products and undesired interruptions in the high-speed production lines causing an inefficient production and an increased cost to manufacture the sanitary articles.

It has surprisingly been found by the present inventors that these problems may be overcome or at least alleviated by a combination of using slot coating for providing the adhesive regions on the fastening wings and using a common release paper having a release force value within the range of from 5 to 30 N/m. The release force is measured according to the Release Force Method as disclosed herein below. These are rather high values, as conventionally the release force value for release papers used when slot coating is below 5 N/m. An increase of the release force value of the release paper, which may be achieved by using less silicone on the release paper, gives a higher fixation, and surprisingly also a reduced risk of unattached portions, such as unattached corner portions, to get caught in the air flow in the machine.

The first adhesive region may have a first adhesive region frontmost edge and the second adhesive region may have a second adhesive region frontmost edge, and a width of the first adhesive region at the first adhesive region frontmost edge may be wider than a width of the second adhesive region at the second adhesive region frontmost edge, the width being measured in the transverse direction.

The width of the first adhesive region at the first adhesive region frontmost edge may be within the range of 25% to 90%, such as 50% to 90%, wider than a width of the second adhesive region at the second adhesive region frontmost edge, the width being measured in the transverse direction.

The first adhesive region frontmost edge and the second adhesive region frontmost edge may be arranged adjacent each other on a common transverse axis, as seen in the transverse direction.

A first adhesive region frontmost edge may alternatively be arranged offset with respect to a second adhesive region frontmost edge, as seen in the longitudinal direction. Optionally, the first adhesive region frontmost edge may be arranged offset in respect to the second adhesive region frontmost edge by at least 5 mm, optionally at least 7 mm, such as from 5 mm to 20 mm, as seen in the longitudinal direction.

Since the first adhesive region frontmost edge is arranged offset with respect to the second adhesive region frontmost edge, as seen in the longitudinal direction, the common release paper may have a corner portion which is not adhesively adhered to the fastening wings this may cause detachment or folding of the release paper or folding of the fastening wing under the release paper. This may in turn result in increased number of defective products and undesired interruptions in the high-speed production lines causing an inefficient production and an increased cost to manufacture the sanitary articles.

However, it has surprisingly been found by the present inventors that these problems may be overcome or at least alleviated by a combination of using slot coating for providing the adhesive regions on the fastening wings and using a common release paper having a Release Force Method as disclosed herein below.

A first adhesive region rearmost edge may be arranged offset with respect to a second adhesive region rearmost edge, as seen in the longitudinal direction. Optionally, the first adhesive region rearmost edge may be arranged offset in respect to the second adhesive region rearmost edge by at least 5 mm, optionally at least 7 mm, such as from 5 mm to 20 mm, as seen in the longitudinal direction

The first and second fastening wings may each be asymmetrical with respect to any transverse axis extending in the transverse direction.

The release paper may have a basis weight within the range of from 32 to 39 g/m², preferably 34-37 g/m². Such release paper, by means of their higher basis weight, has been found to reduce the risk of detachment or folding of the release paper during production.

The first and the second adhesive region may each be a continuous adhesive layer having a basis weight within the range of from 10 to 45 g/m², as measured over the total respective adhesive region. This may provide a stiffening effect on the release paper and has been seen to further alleviate the risk of detachment or folding of the release paper during production, as a consequence of a corner portion being caught in the air flow in the machine.

The common release paper may have a release force value within the range of from 5 to 30 N/m as measured according to the Release Force Method. The release force may preferably be 10-20 N/m as measured according to the Release Force Method.

The common release paper may have a frontmost edge and wherein the frontmost edge of the common release paper is a straight frontmost edge. The common release paper may have a rearmost edge and wherein the rearmost edge of the common release paper is a straight rearmost edge.

The common release paper may have a rectangular shape. A rectangular common release paper is more cost efficient and puts less requirement on positioning of the release paper into the fastening wings.

The first and the second adhesive region may each have a surface area of from 140 to 750 mm². This has been found to be suitably sized adhesive regions for fastening wings and adhesive regions according to the present disclosure.

The adhesive may be a pressure sensitive adhesive.

The first adhesive region frontmost edge may be arranged offset with respect to the second adhesive region frontmost edge with at least 5 mm, as seen in the longitudinal direction. Optionally, the first adhesive region frontmost edge is arranged offset with respect to the second adhesive region frontmost edge with at least 7 mm, such as with at least 10 mm or at least 15 mm offset with respect to the second adhesive region frontmost edge 12a. Optionally, the first adhesive region frontmost edge is arranged offset with respect to the second adhesive region frontmost edge with the range of from 5 mm to 50 mm. The measurements are being done between the frontmost point of the respective adhesive region.

The first adhesive region may have an extension, as seen in the longitudinal direction which is equal or no more than ±15% of an extension of the second adhesive region, as seen in the longitudinal direction.

The present disclosure furthermore relates to a method of attaching a common release paper to a first and a second fastening wing of a sanitary article according to any one of the preceding claims, the method including the steps of;
a) providing a sanitary article including a topsheet and a backsheet, the sanitary article extending in a longitudinal direction and in a transverse direction, the sanitary article having a first and a second longitudinal side edge, a front edge and a rear edge, the sanitary article having a first fastening wing extending from the first longitudinal side edge and a second fastening wing extending from the second longitudinal side edge, the first and second fastening wings being asymmetrical with respect to each other, as seen along any longitudinal axis extending in the longitudinal direction, and folding of the first and the second fastening wings onto the topsheet, the sanitary article being transported in a transport direction corresponding to the longitudinal direction of the sanitary article;
b) providing adhesive on each of the first and the second fastening wing by slot coating, thereby providing a first adhesive region on the first fastening wing, being constituted by the total adhesive area provided on a garment facing side thereof, and a second adhesive region on the second fastening region, being constituted by the total adhesive area provided on a garment facing side thereof, the first coated adhesive region having a first total surface area and the second coated adhesive region having a second total surface area, the first and second coated adhesive regions being asymmetrical with respect to each other, as seen along any longitudinal axis extending in the longitudinal direction and wherein a majority of the first total surface area of the first coated adhesive region is arranged offset with respect to a majority of the second total surface area of the second coated adhesive region, and;
c) applying the common release paper over the first adhesive region on the first fastening wing and the second adhesive region on the second fastening wing, wherein the common release paper has a release force value within the range of from 5 to 30 N/m as measured according to the Release Force Method.

The present disclosure furthermore relates to an alternative method of attaching a common release paper to a first and a second fastening wing of a sanitary article according to any one of the preceding claims, the method including the steps of;
a) providing a sanitary article including a topsheet and a backsheet, the sanitary article extending in a longitudinal direction and in a transverse direction, the sanitary article having a first and a second longitudinal side edge, a front edge and a rear edge, the sanitary article having a first fastening wing extending from the first longitudinal side edge and a second fastening wing extending from the second longitudinal side edge, the first and second fastening wings being asymmetrical with respect to each other, as seen along any longitudinal axis extending in the longitudinal direction, and folding of the first and the second fastening wings onto the topsheet, the sanitary article being transported in a transport direction corresponding to the longitudinal direction of the sanitary article;
b) providing adhesive on a common release paper by slot coating, in a release paper first adhesive region and in a release paper second adhesive region, the common release paper extending in a longitudinal direction, as seen when being applied on the sanitary article, the release paper first adhesive region having a first total surface area and the release paper second adhesive region having a second total area, a majority of the first total surface area of the release paper first adhesive region being arranged offset with respect to a majority of the second total surface area of the release paper second adhesive region, and;
c) applying the common release paper onto the first and the second fastening wing thereby forming a first adhesive region on the first fastening wing, being constituted by the total adhesive area provided on a garment facing side thereof, and a second adhesive region on the second fastening region, being constituted by the total adhesive area provided on a garment facing side thereof, the first coated adhesive region having a first total surface area and the second coated adhesive region having a second total surface area, the first and second coated adhesive regions being asymmetrical with respect to each other, as seen along any longitudinal axis extending in the longitudinal direction and wherein a majority of the first total surface area of the first coated adhesive region is arranged offset with respect to a majority of the second total surface area of the second coated adhesive region, wherein the common release paper has a release force value within the range of from 5 to 30 N/m as measured according to the Release Force Method.

### BRIEF DESCRIPTION OF THE DRAWINGS

The present invention will hereinafter be further explained by means of non-limiting examples with reference to the appended drawings wherein:

FIG. 1 illustrates a sanitary article according to the present disclosure;

FIGS. 2A-2C illustrate a method of attaching a common release paper to a first and a second fastening wing of a sanitary article according to the present disclosure; and

FIG. 3 illustrates step in a method according to the present disclosure wherein the common release paper has been provided with a release paper first and second coated adhesive region prior to applying the common release paper to the fastening wings.

It should be noted that the appended drawings are not necessarily drawn to scale and that the dimensions of some features of the present invention may have been exaggerated for the sake of clarity.

### DETAILED DESCRIPTION

The disclosure will in the following be exemplified by embodiments. It should however be realized that the embodiments are included in order to explain principles of the invention and not to limit the scope of the inventions, defined by the appended claims. Details from two or more embodiments may be combined with each other.

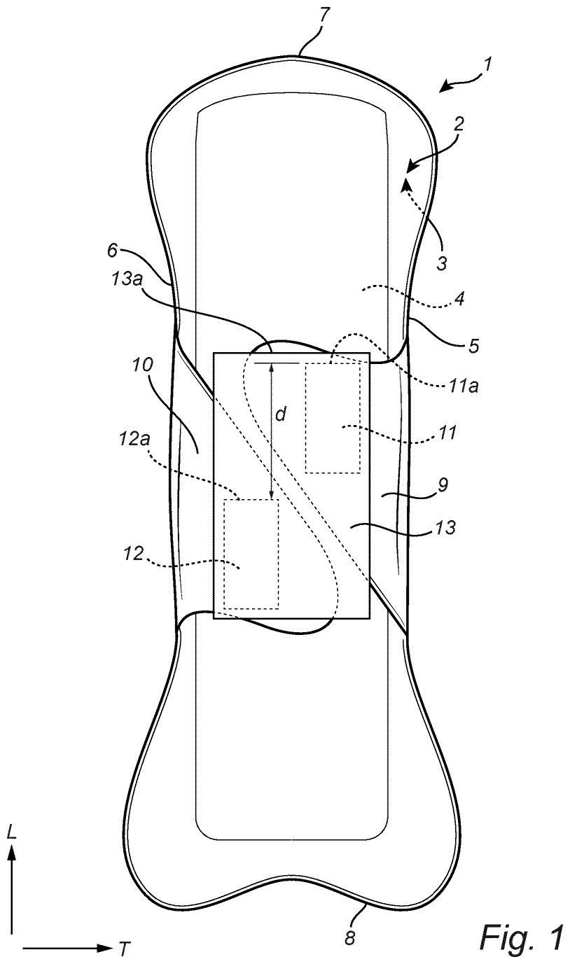

FIG. 1 illustrates a sanitary article 1, here a sanitary napkin. The sanitary article includes a topsheet 2, a backsheet 3 and an absorbent core 4 arranged between the topsheet 2 and the backsheet 3. The sanitary article 1 extends in a longitudinal direction L and in a transverse direction T. The sanitary article 1 has a first longitudinal side edge 5 and a second longitudinal side edge 6, a front edge 7 and a rear edge 8.

The topsheet 2 may include or consist of fibrous nonwoven layer(s) being spunbonded, meltblown, carded, hydroentangled, wetlaid. Suitable nonwoven materials can be composed of natural fibers, such as woodpulp or cotton fibers, synthetic thermoplastic fibers, such as polyolefins, polyesters, polyamides and blends and combinations thereof or from mixtures of natural and synthetic fibers. The materials suited as topshet material should be soft and non-irritating to the skin and be readily penetrated by body fluid, such as menstrual fluid and urine.

The backsheet 3 may consist of a thin plastic film, e.g., a polyethylene or polypropylene film, a nonwoven material coated with a liquid impervious material, a hydrophobic nonwoven, all of which resist liquid penetration. Laminates of plastic films and nonwoven materials may also be used. The backsheet material can be breathable to allow vapor to escape from the absorbent structure, while still preventing liquids from passing through the backsheet material.

An absorbent core 4 constitutes the absorbent structure of the article which acquires and stores bodily fluids. The absorbent core may be of any conventional kind. Examples of commonly occurring absorbent materials are cellulosic fluff pulp, tissue, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form suitable for use in absorbent composites including particles, fibres, flakes, spheres, and the like. A high absorption capacity is provided by using high amounts of superabsorbent material. Thin absorbent cores which are common in sanitary napkins often include a compressed, mixed or layered structure of cellulosic fluff pulp and superabsorbent polymers. The size and absorbent capacity of the absorbent core may be varied to suit different product types, such as sanitary napkins for adult incontinent persons or panty liners.

A suitable technique for manufacturing the absorbent cores of the present disclosure is mat-forming through an air-laying process. In the process an air-permeable mould is provided. Fibrous material is air-laid into the mould and the mould is filled, whereby an absorbent core is produced with a desired amount of fibrous material.

The sanitary article may furthermore include an acquisition layer arranged between the absorbent core and the topsheet and being bonded to the topsheet at least by means of embossings. It may however additionally be adhesively attached to the topsheet. The liquid acquisition layer is adapted to quickly receive and temporarily store discharged liquid before it is absorbed by the absorbent core. Such acquisition distribution layers may be composed of for example airlaid nonwoven, spunlace nonwoven or high loft nonwoven or foam materials. The nonwoven material may be hydrophilic. A hydrophilic material may be obtained by adding a surfactant.

An air laid nonwoven can be produced with fluff, wood pulp, and here the fluff fibres are dispersed into a fast-moving air stream and condensed onto a moving screen by means of pressure and vacuum. The web can be bonded with resin and/or thermal plastic resin dispersed within the pulp. The web can be thermobonded (by heat), latex bonded (with adhesive) or multibonded (a combination of thermo and latex bonding) or mechanically bonded (high compression and temperature, bonding by hydrogen). The basis weight of the airlaid nonwoven can suitably be from 50 to 100 gsm.

A high loft material is a nonwoven material and may be substantially free from absorbing fibres and superabsorbent material. The high loft nonwoven material may include thermoplastic polymer fibres, and may be selected from but not limited to, polyesters, polyamides and polyolefins such as polyethylenes (PE) and polypropylenes (PP), and may be a mixture of any of these. The high loft material refers to low-density bulky fabrics, as compared to flat, paper-like fabrics. High loft webs are characterised by a relatively low density. This means that there is a relatively high amount of void space between the fibres. The high loft nonwoven fibrous layer may typically have a density below 0.200 g/cc (200 kg/m³), in particular ranging from 0.015 g/cc to 0.150 g/cc (15 kg/m³ to 150 kg/m³), in particular from 0.030 g/cc to 0.100 g/cc (30 to 100 kg/m³), for example 0.065 g/cc (65 kg/m³). The average density can be calculated by dividing the basis weight of the high loft layer by its thickness measured at a pressure of 0.5 kPa (see the method details further below). Normally the thickness of high loft materials is more than about 0.5 mm, such as more than 1 mm or suitably 1.5-2.0 mm, and the solid content is low, usually less than 15% by volume. The high loft nonwoven layer may advantageously be a spunmelt nonwoven. Spunmelt is a generic term describing the manufacturing of nonwoven webs directly from thermoplastic polymers. It encompasses two processes and the combination of both: spunlaid (also known as spunbond) nonwoven and meltblown nonwoven. In a spunlaid process, polymer granules are melted and molten polymer is extruded through spinnerets. The continuous filaments are cooled and deposited on to a conveyor to form a uniform web. Some remaining heat can cause filaments to adhere to one another, but this cannot be regarded as the principal method of bonding. The spunlaid process has the advantage of giving nonwovens greater strength, but raw material flexibility is more restricted. Co-extrusion of second components is used in several spunlaid processes, usually to provide extra properties or bonding capabilities. In meltblown web formation, low viscosity polymers are extruded into a high velocity airstream on leaving the spinneret. This scatters the melt, solidifies it and breaks it up into a fibrous web. The liquid acquisition sheet material may be of a spunbonded material and may be a spunbond-meltbond-spunbond (SMS) material. The high loft nonwoven layer may in particular have a thickness ranging from 0.30 mm to 2.00 mm, for example 1.0 mm as measured at a pressure of 0.5 kPa (according to the test method referred to in PCT Application No. PCT/SE2017/050612). The grammage, i.e. basis weight of the high loft material may for example range from 15 gsm to 500 gsm, in particular from 30 gsm to 200 gsm, such as 30-90 gsm, for example 64 gsm.

According to a further variant, the liquid acquisition sheet is a spunlace, also referred to as spunbond, nonwoven material. A spunlace nonwoven product is derived from a process of entangling a web of loose fibres through multiple rows of jets of water at high pressure; this process entangles the fabrics and interlinks the fibres. There are several terms for spunlace nonwoven fabric or spunlaced, such as jet entangled, needled, hydroenentangled or hydraulic, but the term spunlace or spunlaced is the most popular in the nonwoven industry. The raw material for the acquisition sheet can be polypropylene (PP), polyethylene (PE) polyester (PET), polyamide (PA), cellulosic fibres or a combination of these and different weights and compositions are possible, such as viscose, polyester, cotton, nylon and microfibre, wherein viscose is the most commonly used raw material. Thus, if a combination of different fibres is used, this can be a mixture of fibres from different polymers, although each fibre can also include different polymers (e.g., PP/PE bi-component fibres or PP/PE copolymers). The spunlace material usually includes polypropylene or polyethylene fibres which provide for optimal comfort for the nonwoven material. Other suitable fibres for making the nonwoven material are for example natural fibres such as bamboo, cotton and flax. The grammage of the spunlace nonwoven material can be typically from 30-80 gsm.

The sanitary article 1 has a first fastening wing 9 extending from the first longitudinal side edge 5 and a second fastening wing 10 extending from the second longitudinal side edge 6. The first fastening wing 9 includes a first coated adhesive region 11 on a garment facing side thereof and the second fastening wing 10 including a second coated adhesive region 12 on a garment facing side thereof. The first coated adhesive region 11 has a first adhesive region frontmost edge 11a and the second coated adhesive region 12 has a second adhesive region frontmost edge 12a. The adhesive may be a pressure sensitive adhesive.

The sanitary article 1 in FIG. 1 illustrates the article 1 with the first and the second fastening wings 9,10 being arranged in a folded configuration onto the topsheet (2) in a non-overlapping manner, i.e., wherein the first and the second fastening wings 9,10 are not overlapping each other.

The first fastening wing 9 and the second fastening wings 10 are asymmetrical with respect to each other, as seen along any longitudinal axis extending in the longitudinal direction L. In this FIG. 1, the first and second fastening wings 9,10 are additionally each asymmetrical with respect to any transverse axis extending in the transverse direction T, this feature is however an optional feature.

The first adhesive region frontmost edge 11a is arranged offset with respect to the second adhesive region frontmost edge 12a, as seen in the longitudinal direction L, such that the first adhesive region frontmost edge 11a is arranged frontwards with respect to the second adhesive region frontmost edge 12a. The first adhesive region frontmost edge 11a may be arranged offset with respect to the second adhesive region frontmost edge 12a with a distance d of at least 5 mm, as seen in the longitudinal direction L, or optionally with a distance d of at least 7 mm, such as with at least 10 mm or at least 15 mm offset with respect to the second adhesive region frontmost edge 12a. Optionally, the first adhesive region frontmost edge 11a is arranged offset with respect to the second adhesive region frontmost edge 12a with a distance d within the range of from 5 mm to 50 mm.

The first and the second coated adhesive region 11,12 are each a continuous adhesive layer having a respective basis weight within the range of from 10 to 45 g/m². Alternatively, the first and the second coated adhesive region 11,12 may be discontinuously coated, such as with a striped pattern.

In FIG. 1, the first and the second coated adhesive regions 11,12 are depicted as rectangular regions, however, these regions may have any suitable shape. The first and the second coated adhesive regions 11,12 may for examples have the shapes of parallelograms.

The first adhesive region 11 may have an extension, as seen in the longitudinal direction L which is equal or no more than ±15% of an extension of the second adhesive region 12, as seen in the longitudinal direction L. In this FIG. 1, the first and the second coated adhesive regions 11,12 have the same extension in the longitudinal direction L and in the transverse direction T, this is however an optional feature.

The first coated adhesive region 11 and the second coated adhesive region 12 are covered by a common release paper 13. The common release paper 13 has a release force value within the range of from 5 to 30 N/m as measured according to the Release Force Method, preferably 20-30 N/m. The release paper 13 is releasably adhered to the adhesive bearing fastening wings 9,10. The common release paper 13 has a frontmost edge 13a being a straight frontmost edge and has a rectangular shape.

The release paper is coated with silicone and has a basis weight within the range of from 32 to 39 g/m2. The release paper may be coated with silicon in an amount of below 1 gsm, as measured on the side facing the adhesive. The release paper may optionally be coated on both sides with silicone. A suitable release paper is Silcaform 36 GSM K, from Pasaco.

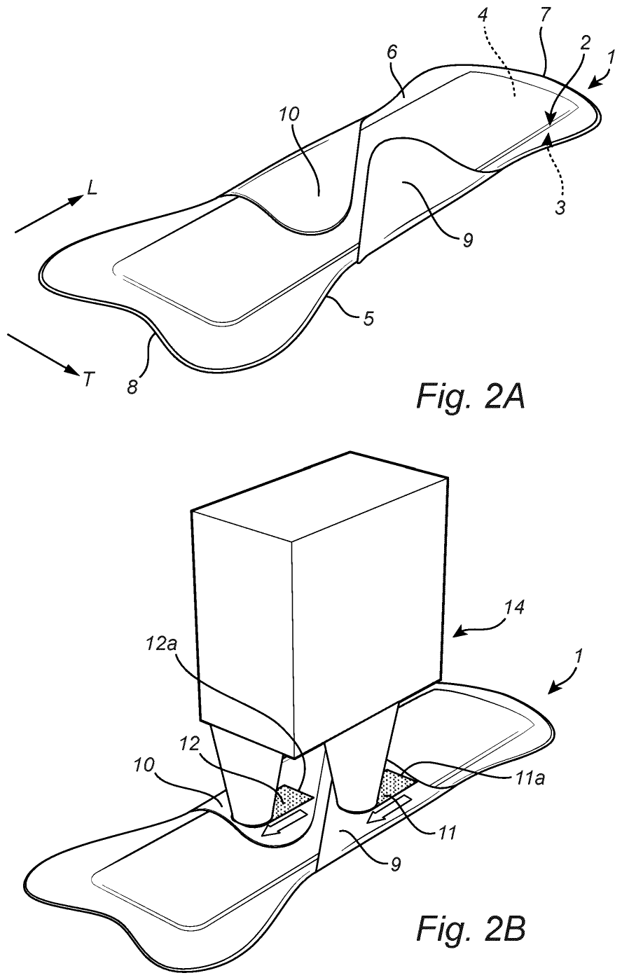

FIGS. 2A-2C illustrate a method of attaching a common release paper 13 (shown in FIG. 2C) to a first and a second fastening wing 9,10 of a sanitary article 1 according to any one of the preceding claims.

FIG. 2A illustrates a first step of providing the sanitary article 1 including a topsheet 2, a backsheet 3 and an absorbent core 4 arranged between the topsheet 2 and the backsheet 3, the sanitary article 1 extending in a longitudinal direction L and in a transverse direction T. The sanitary article 1 has a first and a second longitudinal side edge 5,6, a front edge 7 and a rear edge 8, the sanitary article 1 having a first fastening wing 9 extending from the first longitudinal side edge 5 and a second fastening wing 10 extending from the second longitudinal side edge 6, the first and second fastening wings 9,10 being asymmetrical with respect to any transverse axis extending in the transverse direction T and with respect to each other, as seen along any longitudinal axis extending in the longitudinal direction. In this step, the first and the second fastening wings 9,10 are folded into a folded configuration onto the topsheet 2 in a non-overlapping manner, the sanitary article 1 being transported (not shown) in a transport direction corresponding to the longitudinal direction L of the sanitary article 1.

FIG. 2B illustrates the step of providing adhesive by slot coating, here at a slot coating station 14, to the first and the second fastening wing 9,10, thereby providing a first coated adhesive region 11 on the first fastening wing 9 and a second coated adhesive region 23 on the second fastening wing 10. The first coated adhesive region 11 has a first adhesive region frontmost edge 11a and the second coated adhesive region 12 has a second adhesive region frontmost edge 12a, the first adhesive region frontmost edge 11a being arranged offset with respect to the second adhesive region frontmost edge 12a, as seen in the longitudinal direction L,

FIG. 2C illustrates the final step of applying the common release paper 13 over the first coated adhesive region 11 on the first fastening wing 9 and the second coated adhesive region 12 on the second fastening wing 10. The common release paper 13 applied over the first and second coated adhesive region 11,12 has a release force value within the range of from 5 to 30 N/m as measured according to the Release Force Method. This has been found to significantly reduced the number of defective products and undersired interruptions in high-speed production lines caused by unattached corner portions of the release paper, such as corner portion 13c, being caught in the air flow in the machine, resulting in detachment or folding of the release paper, or folding of the fastening wing under the release paper.

Alternatively, the method may include to provide adhesive by slot coating on the common release paper in a release paper first adhesive region and in a release paper second adhesive region and subsequently apply the release paper provided with the release paper first and second adhesive region thus providing a respective first and second coated adhesive regions as illustrated in FIGS. 1 and 2C.

The adhesive may be applied in an amount within the range of from 10 to 45 g/m² in the respective first and second coated adhesive region 11,12. The common release paper 13 may have a basis weight within the range of from 32 to 39 g/m². The common release paper 13 may have a release force value within the range of from 5 to 30 N/m as measured according to the Release Force Method as disclosed herein below.

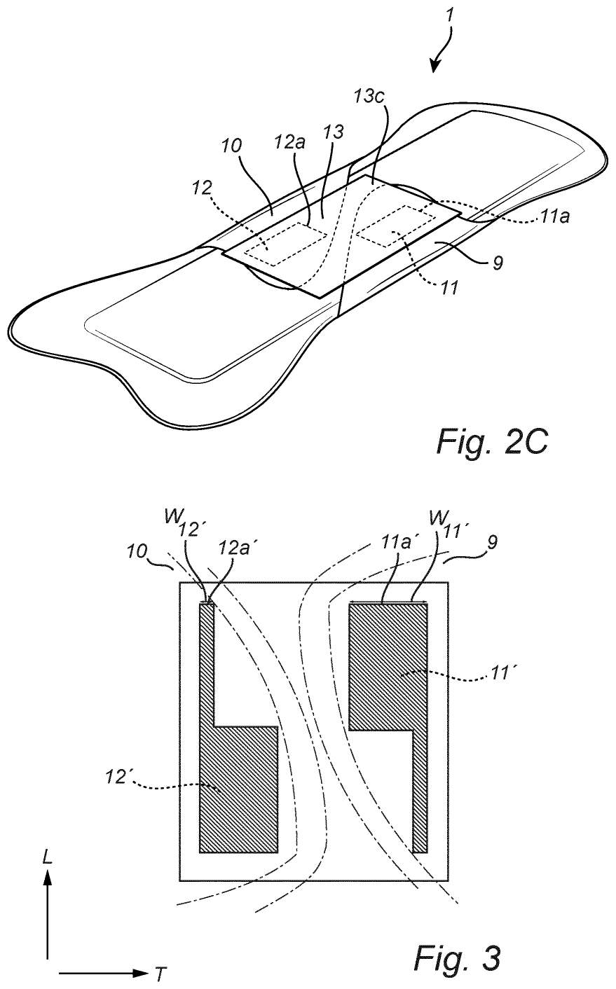

FIG. 3 illustrates a step in an alternative method wherein the adhesive, provided by slot coating, has been applied on a common release paper 14 in a release paper first coated adhesive region 11′ and in a release paper second coated adhesive region 12′, the common release paper 14 extending in a longitudinal direction L, as seen when being applied on the sanitary article 1 (see FIG. 1). The release paper first coated adhesive region 11′ has a first surface area and the release paper second coated adhesive region 12′ having a second surface area, and the first and second coated adhesive regions 11′,12′ being asymmetrical with respect to each other, as seen in the longitudinal direction L, and the majority of the surface area of the release paper first coated adhesive region 11′ is arranged offset with respect to a majority of the surface area of the release paper second coated adhesive region 12′, as seen in the longitudinal direction L. The first coated adhesive region 11′ has a first adhesive region frontmost edge 11a′ and the second coated adhesive region 12′ has a second adhesive region frontmost edge 12a′ and wherein a width W_{11′} of the first adhesive region 11′ at the first adhesive region frontmost edge 11a′ is within the range of from 25% to 90% wider than the width W_{12′} of the second adhesive region 12′, as measured at the second adhesive region frontmost edge 11a′ as measured in the transverse direction T.

The release paper first adhesive region frontmost edge 11a′ and the release paper second adhesive region frontmost edge 12b′ are arranged adjacent each other on a common transverse axis extending in the transverse direction T.

### Release Force Method

The following is a disclosure of the Release Force Method. The Release Force Method is used to measure the release force (N/m) described and claimed herein and. Any reference to the release force mentioned herein is measured by the following method:

The release force of the release paper is evaluated against a standard tape with designation 50110, manufactured by the Tesa company (Hamburg, Germany). The tape is 25 mm wide, 0.3 mm thick, and consists of a 145 mesh acrylate coated fabric with natural rubber as adhesive material. When used for testing, the tape should be maximum one year old. The tape is stored in the dark in a climate of approximately 23° C. and 50% relative humidity.

The release paper is removed from the sanitary article. The paper is then conditioned for at least 2 hours in a laboratory environment set to 23° C. +/−1° C. and 50% rh +/−5% (subsequent testing takes place in this same environment).

For preparing a test sample, the side of the release paper that faces the adhesive region on the sanitary article is aligned against the sticky side of the 25 mm wide tape, gently and without undue pressure. The length direction of the tape should coincide with the longitudinal direction of the release paper (identical to the longitudinal direction of the sanitary article). About one centimeter of the tape protrudes outside of the longitudinal edge of the release paper, and a strip of conventional office printing paper (25 mm wide and about 10 cm long) is attached onto the protruding tape (as a grip area for the subsequent insertion into the tensile tester clamps).

A sample, prepared as above, is then placed with the tape side upwards on a flat, smooth, and hard surface. A uniform pressure of 8.3 kPa (85 g/cm²), suitably from a metal weight with a flat bottom, is then applied over the 25 mm wide tape for 20 hours. The pressure is then removed, and the sample rests unloaded for 4 more hours before testing.

A conventional tensile tester (such as available from the Zwick/Roell, Instron or Lloyd companies), equipped with a sensitive load cell (suitably 10 N) and two vertically aligned clamps (wider than 25 mm) is used for the test. At the longitudinal edge of the release paper, about 10 mm of the release paper is opened up (detached) from the tape for insertion into the lower clamp of the tensile tester. A fixation plate is placed behind (and together with) the sample in the lower clamp (this can be a metal plate about 1 mm thick and at least as high and wide as tested sample). The fixation plate keeps the non-delaminated part of the sample in a vertical position during testing, providing a 180° angle for the delamination interface. The office paper strip (extending into the tape) is inserted into the upper grip. When inserting the sample into the grips, excessive slack or tension over the sample should be avoided. The weight of the sample is tared (zeroed), and the upper grip is then set to move at a speed of 300 mm/min.

The release paper should delaminate (separate) completely from the tape. The encountered force, in Newtons, is continuously registered throughout the delamination. A mean force (arithmetic average) is then calculated for the close sequence of measurement points along the delaminated distance. In this, the first and the last 10 mm of delamination should be disregarded to minimize effects from sample distortions in the two extreme positions. Finally, the output from the test (Newtons for the 25 mm tested width) is reported on a Newton per 1 meter width (N/m) basis, according to the formula below.

$$N / m = \frac{N / 25 mm}{25} \times 1000$$

A release force for the release paper in the context of this disclosure is the arithmetic mean from a measurement of 10 individual release papers (i.e. a mean from the means obtained from the individual samples). The release papers are taken from representative sanitary articles. The release force can also be measured on release paper taken directly from the release paper roll.

1. A sanitary article, such as a sanitary napkin or a pantiliner, the sanitary article comprising a topsheet and a backsheet (3), the sanitary article (1) extending in a longitudinal direction and in a transverse direction, the sanitary article having a first and a second longitudinal side edge, a front edge and a rear edge, the sanitary article having a first fastening wing extending from the first longitudinal side edge and a second fastening wing extending from the second longitudinal side edge, the first fastening wing comprising a first coated adhesive region, being constituted by the total adhesive area provided on a garment facing side thereof and the second fastening wing comprising a second coated adhesive region, being constituted by the total adhesive area provided on a garment facing side thereof, the first coated adhesive region having a first total surface area and the second coated adhesive region having a second total surface area, the first and the second fastening wings being arranged in a folded configuration onto the topsheet and the first and the second coated adhesive regions being covered with a common release paper, wherein the first and second fastening wings are asymmetrical with respect to each other, as seen along any longitudinal axis extending in the longitudinal direction, and in that the first and second coated adhesive regions are asymmetrical with respect to each other, as seen along any longitudinal axis extending in the longitudinal direction, and in that a majority of the first total surface area of the first coated adhesive region is arranged offset with respect to a majority of the second total surface area of the second coated adhesive region, as seen in the longitudinal direction, and wherein the common release paper has a release force value within the range of from 5 to 30 N/m as measured according to the Release Force method.

2. The sanitary article according to claim 1, wherein the first and second fastening wings each are asymmetrical with respect to any transverse axis extending in the transverse direction.

3. The sanitary article according to claim 1, wherein the first coated adhesive region has a first adhesive region frontmost edge and the second coated adhesive region has a second adhesive region frontmost edge and wherein a width of the first adhesive region at the first adhesive region frontmost edge is wider than a width of the second adhesive region at the second adhesive region frontmost edge, as measured in the transverse direction.

4. The sanitary article according to claim 3, wherein a width of the first adhesive region (11) at the first adhesive region frontmost edge, is within the range of from 25% to 90% wider than the width of the second adhesive region, as measured at the second adhesive region frontmost edge as measured in the transverse direction.

5. The sanitary article according to claim 3, wherein the first adhesive region frontmost edge and the second adhesive region frontmost edge are arranged adjacent each other on a common transverse axis extending in the transverse direction.

6. The sanitary article according to claim 1, wherein the first coated adhesive region has a first adhesive region frontmost edge and the second coated adhesive region has a second adhesive region frontmost edge and wherein the first adhesive region frontmost edge is arranged offset with respect to the second adhesive region frontmost edge, as seen in the longitudinal direction.

7. The sanitary article according to claim 6 wherein the first adhesive region frontmost edge is arranged offset with respect to the second adhesive region frontmost edge with at least 5 mm, as seen in the longitudinal direction.

8. The sanitary article according to claim 1, wherein the first coated adhesive region has a first adhesive region rearmost edge and the second coated adhesive region has a second adhesive region rearmost edge and wherein the first adhesive region rearmost edge is arranged offset with respect to the second adhesive region rearmost edge.

9. The sanitary article according to claim 8, wherein the first adhesive region rearmost edge is arranged offset with respect to the second adhesive region rearmost edge with at least 5 mm, as seen in the longitudinal direction.

10. The sanitary article according to claim 1, wherein the common release paper has a basis weight within the range of from 32 to 39 g/m².

11. The sanitary article according to claim 1, wherein the first and the second coated adhesive region each are a continuous adhesive layer having a basis weight within the range of from 10 to 45 g/m².

12. The sanitary article according to claim 1, wherein the common release paper has a release force within the range of from 10 to 20 N/m as measured according to the Release Force Method.

13. The sanitary article according to claim 1, wherein the common release paper has a rectangular shape.

14. The sanitary article according to claim 1, wherein the first adhesive region has an extension, as seen in the longitudinal direction which is equal or no more than ±15% of an extension of the second adhesive region, as seen in the longitudinal direction.

15. A method of attaching a common release paper to a first and a second fastening wing of a sanitary article according to claim 1, the method including the steps of;
a) providing a sanitary article comprising a topsheet and a backsheet, the sanitary article extending in a longitudinal direction and in a transverse direction, the sanitary article having a first and a second longitudinal side edge, a front edge and a rear edge, the sanitary article having a first fastening wing extending from the first longitudinal side edge and a second fastening wing extending from the second longitudinal side edge, the first and second fastening wings being asymmetrical with respect to each other, as seen along any longitudinal axis extending in the longitudinal direction, and folding the first and the second fastening wings in a folded configuration onto the topsheet, the sanitary article being transported in a transport direction corresponding to the longitudinal direction of the sanitary article;
b) providing adhesive by slot coating on the first and the second fastening wing, thereby providing a first coated adhesive region on the first fastening wing, being constituted by the total adhesive area provided on the first fastening wing on a garment facing side thereof and a second coated adhesive region on the second fastening wing, being constituted by the total adhesive area provided on the first fastening wing on a garment facing side thereof, the first coated adhesive region having a first total surface area and the second coated adhesive region having a second total surface area, the first and second coated adhesive regions being asymmetrical with respect to each other, as seen in the longitudinal direction, and wherein a majority of the first total surface area of the first coated adhesive region is arranged offset with respect to a majority of the second total surface area of the second adhesive region, as seen in the longitudinal direction, and;
c) applying a common release paper over the first coated adhesive region on the first fastening wing and the second coated adhesive region on the second fastening wing, wherein the common release paper has a release force value within the range of from 5 to 30 N/m as measured according to the Release Force Method.

16. A method of attaching a common release paper to a first and a second fastening wing of a sanitary article according to claim 1, the method including the steps of;
a) providing a sanitary article comprising a topsheet and a backsheet, the sanitary article extending in a longitudinal direction and in a transverse direction, the sanitary article having a first and a second longitudinal side edge, a front edge and a rear edge, the sanitary article having a first fastening wing extending from the first longitudinal side edge and a second fastening wing extending from the second longitudinal side edge, the first and second fastening wings being asymmetrical with respect to each other, as seen along any longitudinal axis extending in the longitudinal direction, and folding the first and the second fastening wings in a folded configuration onto the topsheet, the sanitary article being transported in a transport direction corresponding to the longitudinal direction of the sanitary article;
b) providing adhesive by slot coating on a common release paper in a release paper first adhesive region and in a release paper second adhesive region, the common release paper extending in a longitudinal direction, as seen when being applied on the sanitary article, the release paper first adhesive region having a first surface area and the release paper second adhesive region having a second surface area, and wherein the first and second coated adhesive regions are asymmetrical with respect to each other, as seen in the longitudinal direction, and the majority of the surface area of the first coated adhesive region is arranged offset with respect to a majority of the surface area of the second adhesive region, as seen in the longitudinal direction, and;
c) applying the common release paper over the first and the second fastening wing thereby forming a first coated adhesive region being constituted by the total adhesive area provided on the first fastening wing and a second coated adhesive region on the second fastening wing, the first coated adhesive region the first fastening wing on a garment facing side there of having the first surface area and the second coated adhesive region having the second surface area, the first and second coated adhesive regions being asymmetrical with respect to each other, as seen in the longitudinal direction, and wherein a majority of the surface area of the first coated adhesive region is arranged offset with respect to a majority of the second surface area of the second adhesive region, as seen in the longitudinal direction, wherein the common release paper has a release force value within the range of from 5 to 30 N/m as measured according to the Release Force Method.
